(19) United States
(12) Patent Application Publication
(10) Pub. No.: US 2026/0248870 A1
(43) Pub. Date: Aug. 27, 2026

(54) METHOD FOR IMPROVING SKIN ELASTICITY USING A COMBINATION OF EXTRACTED PRODUCT OF ARONIA MELANOCARPA FRUIT AND BIFIDOBACTERIUM LONGUM CB108 POSTBIOTICS

(71) Applicant: CHAMBIO CO., LTD., Taichung City (TW)

(72) Inventors: Hung-Pin CHIU, Taichung City (TW); Yu-Ting LIN, Taichung City (TW)

(21) Appl. No.: 19/245,654

(22) Filed: Jun. 23, 2025

(30) Foreign Application Priority Data
Feb. 21, 2025 (TW) 114106564

(51) Int. Cl. A61K 36/73; A61K 35/00; A61P 17/00; C12N 1/20

(52) U.S. Cl. CPC A61K 36/73; A61P 17/00; C12N 1/20; A61K 2035/11; A61K 2236/333

(57) ABSTRACT

A method for improving skin elasticity includes use of a composition containing an ethanol-extracted product of Aronia melanocarpa fruit and Bifidobacterium longum CB108 which is deposited at the Leibniz Institute DSMZ-German Collection of Microorganisms and Cell Cultures GmbH under an accession number DSM 33895 in accordance with the Budapest Treaty.

### CROSS-REFERENCE TO RELATED APPLICATION

This application claims priority to Taiwanese Invention patent application No. 114106564, filed on Feb. 21, 2025, the entire disclosure of which is incorporated by reference herein.

### SEQUENCE LISTING XML

The Sequence Listing submitted concurrently herewith with a file name of “PE-71829-AM-SEQUENCE LISTING.xml,” a creation date of May 16, 2025, and a size of 9.45 kilobytes, is part of the specification and is incorporated by reference in its entirety.

### FIELD

The present disclosure relates to a method for improving skin elasticity using a composition including an ethanol-extracted product of Aronia melanocarpa fruit and Bifidobacterium longum CB108.

### BACKGROUND

Dermal fibroblasts play a crucial role in maintaining skin elasticity by producing extracellular matrix (ECM) which includes collagen and elastin (ELN) to provide structural support, and by secreting inhibitors of matrix metalloproteinases (MMPs) which include TIMP metallopeptidase inhibitor 1 (TIMP-1) to slow down degradation of the ECM by the MMPs.

In recent years, demand for improving skin elasticity has been increasing, especially the use of natural ingredients that do not cause undesirable side effect on the skin, which has become a trend in today's market. Therefore, researches have been trying to identify safe active components from microorganisms or plants to meet the increasing demand of the broad market.

Aronia melanocarpa (Latin name), commonly known in English as black chokeberry, is a deciduous shrub of the genus Aronia in the family Rosaceae, and is native to North America and has been introduced to Europe. The primary utilization part of the Aronia melanocarpa is fruit thereof, which has been recognized to have antioxidant and metabolic disease improvement effects.

It has been reported in Lee H R et al., (2022), Oxid Med Cell Longev., 2022:4392256 that an ethanol-extracted product of Aronia melanocarpa could promote collagen production by the dermal fibroblasts and inhibit expression of the MMPs.

In view of the aforesaid, there is still a need to develop an effective way for improving skin elasticity.

### SUMMARY

Therefore, an object of the present disclosure is to provide a method for improving skin elasticity, which can alleviate at least one of the drawbacks of the prior art, and which includes administering to a subject in need thereof a composition including an ethanol-extracted product of Aronia melanocarpa fruit and Bifidobacterium longum CB108.

The Bifidobacterium longum CB108 is deposited at the Leibniz Institute DSMZ-German Collection of Microorganisms and Cell Cultures GmbH under an accession number DSM 33895 in accordance with the Budapest Treaty.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the relative expression level of the COL3A1 gene determined in each group of Example 1, infra, in which the symbol “*” represents p<0.05 (compared with the control group), and the symbol “#” represents p<0.05 (compared with the comparative group 1).

FIG. 2 shows the relative expression level of the TIMP-1 gene determined in each group of Example 1, infra, in which the symbol “*” represents p<0.05 (compared with the control group), the symbol “#” represents p<0.05 (compared with the comparative group 1), and the symbol “$” represents p<0.05 (compared with the comparative group 2).

FIG. 3 shows the relative expression level of the ELN gene determined in each group of Example 1, infra, in which the symbol “*” represents p<0.05 (compared with the control group), the symbol “#” represents p<0.05 (compared with the comparative group 1), and the symbol “$” represents p<0.05 (compared with the comparative group 2).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for improving skin elasticity, which includes administering to a subject in need thereof a composition including an extracted product of Aronia melanocarpa fruit and Bifidobacterium longum CB108. To be specific, the extracted product of Aronia melanocarpa fruit is an ethanol-extracted product of Aronia melanocarpa fruit.

The Bifidobacterium longum CB108 is deposited at the Leibniz Institute DSMZ-German Collection of Microorganisms and Cell Cultures GmbH under an accession number DSM 33895 in accordance with the Budapest Treaty.

As used herein, the term “administering” can be used interchangeably with other terms such as “administration”, and means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

According to the present disclosure, a weight ratio of the ethanol-extracted product of Aronia melanocarpa fruit to the Bifidobacterium longum CB108 ranges from 1:0.01 to 1:100. In certain embodiments, a weight ratio of the ethanol-extracted product of Aronia melanocarpa fruit to the Bifidobacterium longum CB108 ranges from 1:0.4 to 1:50. In an exemplary embodiment, a weight ratio of the ethanol-extracted product of Aronia melanocarpa fruit to the Bifidobacterium longum CB108 is 1:0.4. In another exemplary embodiment, a weight ratio of the ethanol-extracted product of Aronia melanocarpa fruit to the Bifidobacterium longum CB108 is 1:50.

According to the present disclosure, the Bifidobacterium longum CB108 may be viable or non-viable bacterial cells, concentrated or non-concentrated, a liquid, a paste, a semi-solid, or a solid (e.g., a pellet, a granule, or a powder), and may be heat-killed, frozen, dried, freeze-dried, or spray/fluid bed-dried. In certain embodiments, the Bifidobacterium longum CB108 is heat-killed and is present in a form of a spray-dried powder which serves as a postbiotic component.

According to the present disclosure, the Bifidobacterium longum CB108 may be prepared as a bacterial powder having a total bacterial concentration ranging from 10⁷ cells/g to 10¹³ cells/g. In certain embodiments, the Bifidobacterium longum CB108 may be prepared as a bacterial powder having a total bacterial concentration ranging from 10¹⁰ cells/g to 10¹² cells/g. In an exemplary embodiment, the Bifidobacterium longum CB108 may be prepared as a bacterial powder having a total bacterial concentration of 10¹¹ cells/g.

As used herein, the term “heat-killed” can be used interchangeably with other terms such as “heat-inactivated”, and refers to subjecting probiotics to a heat treatment for a predetermined time period so as to kill them.

According to the present disclosure, the heat-killed Bifidobacterium longum CB108 may be prepared using techniques well-known to those skilled in the art. In this regard, those skilled in the art may refer to journal articles, e.g., Segawa S. et al. (2008), Int. J. Food Microbiol., 128:371-377, and Ben Othman M. et al. (2020), Food Res. Int., doi: 10.1016/j.foodres.2019.108792.

According to the present disclosure, the heat-killed Bifidobacterium longum CB108 may be prepared by subjecting the Bifidobacterium longum CB108 to a heat treatment at a temperature ranging from 60° C. to 140° C. for a time period ranging from 1 second to 30 minutes. In an exemplary embodiment, the heat treatment is performed at a temperature of 73±2° C. for a time period of 15 seconds.

According to the present disclosure, the term “Aronia melanocarpa fruit” refers to a fruit that contains pulp (i.e., edible parts) of the Aronia melanocarpa. In certain embodiments, the Aronia melanocarpa fruit further includes peel and seeds of the Aronia melanocarpa.

According to the present disclosure, the ethanol-extracted product of Aronia melanocarpa fruit may be obtained as a commercial product, or may be prepared using techniques well-known to those skilled in the art.

It should be noted that the operating conditions for conducting an ethanol extraction treatment of the Aronia melanocarpa fruit may be adjusted based on factors such as the processing method of the Aronia melanocarpa fruit and a weight ratio of ethanol to the Aronia melanocarpa fruit, so as to achieve the best extraction results.

According to the present disclosure, Aronia melanocarpa fruit harvested from various regions which is selected from the group consisting of the United States, India, Europe, China, and combinations thereof may subjected to the ethanol extraction treatment. In an exemplary embodiment, Aronia melanocarpa fruit harvested from Poland is subjected to the ethanol extraction treatment.

According to the present disclosure, the Aronia melanocarpa fruit subjected to the ethanol extraction treatment may be fresh, or may be prepared by a treatment selected from the group consisting of a drying treatment, a grinding treatment, a chopping treatment, a comminuting treatment, and combinations thereof. In certain embodiments, before the ethanol extraction treatment, the Aronia melanocarpa fruit may be subjected to the comminuting treatment using techniques well-known to those skilled in the art.

According to the present disclosure, the ethanol extraction treatment of the Aronia melanocarpa fruit may be performed with a weight ratio of the Aronia melanocarpa fruit to an ethanol solution ranging from 1:1 to 1:20. In certain embodiments, the ethanol extraction treatment of the Aronia melanocarpa fruit may be performed with a weight ratio of the Aronia melanocarpa fruit to the ethanol solution ranging from 1:3 to 1:8. In an exemplary embodiment, the ethanol extraction treatment of the Aronia melanocarpa fruit is performed with a weight ratio of the Aronia melanocarpa fruit to the ethanol solution of 1:5.

According to the present disclosure, the ethanol solution may have an ethanol concentration ranging from 0.01% to 95%. In an exemplary embodiment, the ethanol solution has an ethanol concentration of 40%.

According to the present disclosure, the ethanol extraction treatment of the Aronia melanocarpa fruit may be performed at a temperature ranging from 40° C. to 100° C. In certain embodiments, the ethanol extraction treatment of the Aronia melanocarpa fruit may be performed at a temperature ranging from 75° C. to 95° C. In an exemplary embodiment, the ethanol extraction treatment of the Aronia melanocarpa fruit is performed at a temperature of 85° C.

According to the present disclosure, the ethanol extraction treatment of the Aronia melanocarpa fruit may be performed for a time period ranging from 0.5 hours to 3 hours. In an exemplary embodiment, the ethanol extraction treatment of the Aronia melanocarpa fruit is performed for a time period of 2 hours.

According to the present disclosure, the ethanol-extracted product of Aronia melanocarpa fruit and the Bifidobacterium longum CB108 may be co-administered to a subject in need of improving skin elasticity. In certain embodiments, the ethanol-extracted product of Aronia melanocarpa fruit and the Bifidobacterium longum CB108 may be formulated into a single dosage form or separate dosage forms, and the single dosage form or the separate dosage forms may be administered simultaneously. In certain embodiments, the ethanol-extracted product of Aronia melanocarpa fruit and the Bifidobacterium longum CB108 may be formulated into separate dosage forms, and the two separate dosage forms may be administered alternately or sequentially, with a predetermined time interval between administrations. In certain embodiments, a length of the predetermined time interval may be adjusted as required to allow pharmacological effects of the ethanol-extracted product of Aronia melanocarpa fruit and the Bifidobacterium longum CB108 to either overlap or non-overlap over time within the subject. In an exemplary embodiment, the ethanol-extracted product of Aronia melanocarpa fruit and the Bifidobacterium longum CB108 are formulated into a single dosage form.

According to the present disclosure, the composition may be formulated as a food product using a standard technique well-known to one of ordinary skill in the art. For example, the composition may be formulated in the form of a food additive, which is added to an edible material to prepare a food product for human or animal consumption.

As used herein, the term “food product” refers to any article or substance that can be ingested by a subject into the body thereof. Examples of the food product may include, but are not limited to, health foods, dietary supplements, milk powders, fermented milk, yogurt, butter, beverages (e.g., tea, coffee, etc.), functional beverages, a flour product, baked foods, confectionery, candies, fermented foods, and animal feeds.

According to the present disclosure, the food product may further include a food additive widely employed in the art of food-manufacturing. Examples of the food additive may include, but are not limited to, a starch, a dextrin, lactose, maize flours, rice flours, tricalcium phosphate, silicon dioxide, magnesium stearate, calcium carbonate, glucose, sucrose, fructose, a sugar alcohol, an oligosaccharide, a sugar substitute, fruit juice powders, yeast powders, skim milk powders, casein, whey protein, amino acid, citric acid, citrate, lactic acid, lactate and nucleotide.

According to the present disclosure, the composition may be prepared in the form of a pharmaceutical composition. The pharmaceutical composition may be formulated into a dosage form suitable for oral or topical administration using technology well known to those skilled in the art.

According to the present disclosure, examples of the dosage form suitable for oral administration may include, but are not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well-known to those skilled in the art. Examples of the external preparation may include, but are not limited to, emulsions, gels, ointments, creams, patches, liniments, powders, aerosols, sprays, lotions, serums, pastes, foams, drops, suspensions, salves, and bandages.

According to the present disclosure, the external preparation is prepared by mixing the composition with a base which is well-known to those skilled in the art.

According to the present disclosure, the base may include one or more of the following additives: water, alcohols, glycols, hydrocarbons (e.g., petroleum jelly and white petrolatum), waxes (e.g., paraffin, and yellow waxes), preserving agents, antioxidants, surfactants, absorption enhancers, stabilizing agents, gelling agents (e.g., Carbopol® 941 NF polymers, microcrystalline celluloses, and carboxymethyl cellulose), active agents, humectants, odor absorbers, spices, pH adjusting agents, chelating agents, emulsifiers, occlusive agents, softening agents, thickening agents, solubilizing agents, penetration enhancers, anti-irritants, colorants, propellants, etc. The choice and amount of the aforesaid additives are within the expertise and routine skills of those skilled in the art.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to present disclosure, the composition may be prepared in the form of a cosmeceutical composition. The cosmeceutical composition may be manufactured to a form suitable for skincare or make-up using technology well-known to those skilled in the art.

According to the present disclosure, examples of the form suitable for skincare or make-up may include, but are not limited to, aqueous solutions, aqueous alcohol solutions or oily solutions, oil-in-water type emulsions, water-in-oil type emulsions or composite type emulsions, gels, ointments, creams, masks, patches, packs, liniments, powders, aerosols, sprays, lotions, serums, pastes, foams, dispersions, drops, suspensions, salves, bandages, mousses, sunblock, skin toners, foundation, eyeshadows, makeup removers, soaps, other body cleansing products, etc.

According to present disclosure, the cosmeceutical composition may further include the aforesaid pharmaceutically acceptable carrier and/or a cosmetically acceptable adjuvant widely employed in the art of cosmetic-manufacturing. For instance, the cosmetically acceptable adjuvant may include one or more of the following agents: solvents, gelling agents, active agents, preservatives, antioxidants, screening agents, chelating agents, surfactants, coloring agents, thickening agents, fillers, spices, and odor absorbers. The choice and amount of the aforesaid agents are within the expertise and routine skills of those skilled in the art.

According to the present disclosure, the dose and frequency of administration of the composition may vary depending on the following factors: the severity of the illness or disorder to be treated, routes of administration, and age, physical condition and response of the subject to be treated. In general, the composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials

1. Bifidobacterium longum CB108

The Bifidobacterium longum CB108, which is known and readily available to the public and which is disclosed in TW I802009 B (counterpart of U.S. Pat. No. 12,090,182 B2), was obtained from the Microbiology Research Laboratory of the Department of Food Science and Biotechnology, National Chung-Hsing University, Taichung, Taiwan, and has been deposited at the Bioresource Collection and Research Center (BCRC) of the Food Industry Research and Development Institute (FIRDI) (No. 331, Shih-Pin Rd., Hsinchu City 300, Taiwan) under an accession number BCRC 910894 on May 8, 2019. In addition, the Bifidobacterium longum CB108 has also been deposited on the International Recognition of the Deposit of Microorganisms for the Purpose of Patent Procedure at the International Depositary Authority, i.e., Leibniz Institute DSMZ-German Collection of Microorganisms and Cell Cultures GmbH (Inhoffenstr. 7B, D-38124 Braunschweig, Germany), under an accession number DSM 33895 in accordance with the Budapest Treaty on Jun. 9, 2021.

2. Preparation of Heat-Killed Bacterial Powder of Bifidobacterium longum CB108

The heat-killed bacterial powder of the Bifidobacterium longum CB108 (abbreviated as Bifidobacterium longum CB108 postbiotics) was prepared with primarily reference to the method described in TW 1802009 B. In brief, the Bifidobacterium longum CB108 as described in section 1 of “General Experimental Materials” was inoculated into a MRS broth (Manufacturer: BD Difco, Cat. no.: DF0881-17-5), and was then cultivated in an incubator (37° C.) for 16 hours to obtain a liquid culture of the Bifidobacterium longum CB108. Next, the liquid culture of the Bifidobacterium longum CB108 was subjected to a heat treatment by high-temperature short-time (HTST) pasteurization at 73±2° C. for 15 seconds, so as to kill the Bifidobacterium longum CB108 in the liquid culture. A bacterial count of the Bifidobacterium longum CB108 in the liquid culture was subsequently determined to adjust a bacterial concentration thereof, followed by conducting a centrifugation treatment, so as to collect the resultant bacterial pellet. Afterwards, the resultant bacterial pellet was subjected to a spray-drying treatment, thereby obtaining the Bifidobacterium longum CB108 postbiotics having the bacterial concentration of 10¹¹ cells/g.

3. Preparation of Ethanol-Extracted Product of Aronia melanocarpa Fruit

First, Aronia melanocarpa fruit (including peel, pulp, and seeds) was washed with deionized water, followed by conducting a comminuting treatment using a homogenizer, so as to obtain a comminuted fruit. Nest, 100 g of the comminuted fruit thus obtained was mixed with 500 ml of 40% ethanol, followed by conducting an ethanol extraction treatment at 85° C. for 120 minutes. The resultant mixture was subsequently filtered to obtain a filtrate, followed by subjecting the filtrate to a drying treatment through vacuum concentration, so as to obtain a powder of the ethanol-extracted product of Aronia melanocarpa fruit.

### Example 1. Evaluation of the Effect of Ethanol-Extracted Product of Aronia melanocarpa Fruit and Bifidobacterium longum CB108 on Promotion of Expression of Extracellular Matrix (ECM)-Related Genes

### Experimental Materials

### 1. Source and Cultivation of Skin Cells

Human dermal fibroblast cell line CCD-966SK (BCRC 60153) used in the following experiments were obtained from the BCRC of the FIRDI. The CCD-966SK cells were grown in a 10-cm Petri dish containing minimum essential medium (MEM, Manufacturer: Sigma, Cat. no.: M4526) supplemented with 10% fetal bovine serum (FBS, Manufacturer: Sigma, Cat. no.: F7524), 1.5 g/L of sodium bicarbonate (Manufacturer: Sigma, Cat. no.: S5761), 1 mM of sodium pyruvate (Manufacturer: Sigma, Cat. no.: P2256), 1 mM of a MEM non-essential amino acid solution (Manufacturer: Sigma, Cat. no.: M7145), and 1% penicillin-streptomycin (Manufacturer: Sigma, Cat. no.: P4333), followed by cultivating in an incubator at 37° C. with 5% CO₂. Subsequently, medium change was performed every two to three days. When the CCD-966SK cells reached approximately 80% to 90% confluence, cell passage was performed.

2. Preparation of Solution of Bifidobacterium longum CB108 Postbiotics

Briefly, an appropriate amount of the Bifidobacterium longum CB108 postbiotics prepared in section 2 of “General Experimental Materials” were dissolved in an appropriate amount of MEM. so as to obtain the solution of Bifidobacterium longum CB108 postbiotics.

3. Preparation of Solution of Ethanol-Extracted Product of Aronia melanocarpa Fruit

Briefly, an appropriate amount of the ethanol-extracted product of Aronia melanocarpa fruit prepared in section 3 of “General Experimental Materials” were dissolved in an appropriate amount of MEM. so as to obtain the solution of ethanol-extracted product of Aronia melanocarpa fruit.

### Experimental Procedures

### A. Drug Administration to Skin Cells

First, the CCD-966SK cells prepared in section 1 of “Experimental Materials” of this example were divided into 4 groups, including a control group, two comparative groups (i.e., comparative groups 1 and 2), and an experimental group. The CCD-966SK cells of each group were seeded at a density of 6×10⁵ cells into a 6-cm Petri dish containing 5 mL of MEM, followed by cultivation in an incubator (37° C., 5% CO₂) for 24 hours. Next, the culture medium in each group was removed to be replaced with a fresh culture medium. Subsequently, the cell culture of each of the comparative groups 1 and 2 and the experimental group were treated with an appropriate amount of the solution of Bifidobacterium longum CB108 postbiotics and/or the solution of ethanol-extracted product of Aronia melanocarpa fruit obtained respectively in section 2 and section 3 of “Experimental Materials” of this example, such that the treating agents had different final concentrations in each of the groups as shown in Table 1 below. In addition, the cell culture of the control group received no treatment. After cultivation in an incubator at 37° C. for 24 hours, the CCD-966SK cells in the cell culture of each group were collected for the subsequent determination as described in section B of “Experimental Procedures” of this example.

TABLE 1

| | Testing agent with a final concentration | |
|---|---|---|
| | (μg/mL) | |
| | Solution of | Solution of ethanol- |
| | Bifidobacterium longum | extracted product of Aronia |
| Group | CB108 postbiotics | melanocarpa fruit |
| Comparative | — | 200 |
| group 1 | | |
| Comparative | 10000 | — |
| group 2 | | |
| Experimental | 5000 | 100 |
| group | | |

### B. Determination of Relative Expression Levels of ECM-Related Genes

First, the CCD-966SK cells of each group collected in section A of “Experimental Procedures” of this example were subjected to total RNA extraction using a Total RNA Isolation Kit (Manufacturer: GeneDirex, Inc., Cat. no.: NA017-0100) in accordance with the manufacturer's instructions. Next, the resultant total RNA of each group was used as a template for synthesizing cDNA by reverse transcription polymerase chain reaction (RT-PCR) using a High-Capacity cDNA Reverse Transcription Kit (Manufacturer: Applied Biosystems, Cat. no.: 4368813) in accordance with the manufacturer's instructions.

Thereafter, the thus obtained cDNA, serving as a DNA template, was subjected to quantitative real-time polymerase chain reaction (quantitative real-time PCR) based on SYBR-Green fluorescence (i.e., a double-stranded DNA-binding dye), which was performed on a StepOne™ real-time PCR system using primer pairs specific for collagen type III α1 (COL3A1) gene, tissue inhibitor of metalloproteinases-1 (TIMP-1) gene, and elastin (ELN) gene, respectively, as shown in Table 2 below and the reaction conditions shown in Table 3 below. In addition, β-actin gene was used as an internal control in the quantitative real-time PCR analysis to normalize the gene expression data (see Table 2).

TABLE 2

| | NCBI | | Nucleotide sequence |
|---|---|---|---|
| Target gene | accession no. | Primer | (5′→3′) |
| COL3A1 | NM_000090 | Forward | accaggagagaagggatcgc |
| | | primer | (SEQ ID NO: 1) |
| | | COL3A1-F | |
| | | Reverse | ttcccctaggacctggcatg |
| | | primer | (SEQ ID NO: 2) |
| | | COL3A1-R | |
| TIMP-1 | NM_003254 | Forward | accagaccaccttataccagcg |
| | | primer | (SEQ ID NO: 3) |
| | | TIMP-1-F | |
| | | Reverse | ggactggaagcccttttcagag |
| | | primer | (SEQ ID NO: 4) |
| | | TIMP-1-R | |
| ELN | M36860 | Forward | ggcctggaggcaaacctctt |
| | | primer | (SEQ ID NO: 5) |
| | | ELN-F | |
| | | Reverse | ccaccaactcctgggacacc |
| | | primer | (SEQ ID NO: 6) |
| | | ELN-R | |
| β-actin | EF036500 | Forward | tcacccacactgtgcccatctacga |
| | | primer | (SEQ ID NO: 7) |
| | | β-actin-F | |
| | | Reverse | cagcggaaccgctcattgccaatgg |
| | | primer | (SEQ ID NO: 8) |
| | | β-actin-R | |

TABLE 3

| Reaction mix | Volume (μL) |
|---|---|
| cDNA (10 ng/μL) | 1 |
| Forward primer (10 μM) | 1 |
| Reverse primer (10 μM) | 1 |
| KAPA SYBR ® FAST qPCR Master Mix | 5 |
| (Manufacturer: KAPA biosystems, Inc.) | |
| Sterile water | 2 |

Operating conditions: initial denaturation at 95° C. for 20 seconds, followed by 40 cycles of denaturation at 90° C. for 30 seconds, and then annealing and extension simultaneously at 60° C. for 30 seconds.

The resultant PCR product was subjected to determination of fluorescence intensity, followed by calculating the cycle threshold (Ct) value of each gene. Quantitative real-time PCR data were analyzed using the comparative Ct method. Briefly, the Ct value of the ECM-related genes in each group was normalized with that of the β-actin gene, and the resultant normalized values of the ECM-related genes in each group were further divided by the resultant normalized values of the ECM-related genes in the control group so as to determine the relative expression levels of the ECM-related genes (i.e., the COL3A1, TIMP-1, and ELN genes).

The data thus obtained were performed in triplicates. The experimental data of all the groups were expressed as mean±standard error of the mean (SEM), and were analyzed using independent sample t-test, so as to evaluate the differences between the groups. Statistical significance is indicated by p<0.05.

### Results

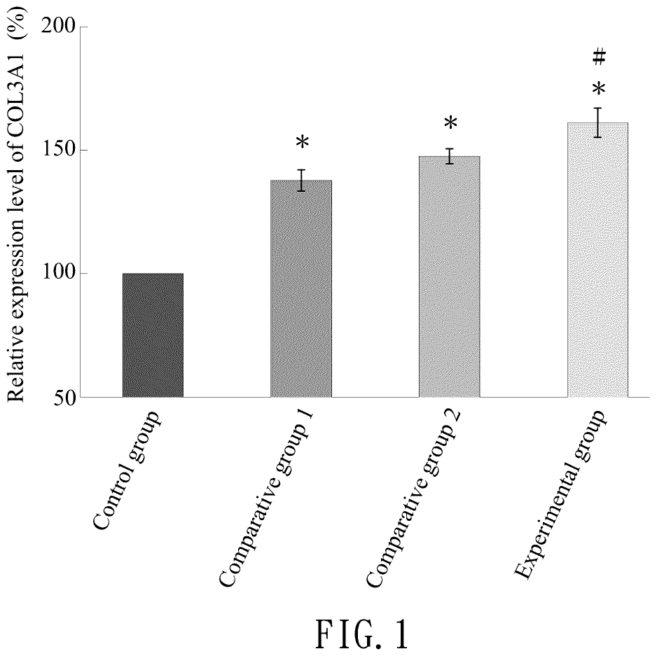

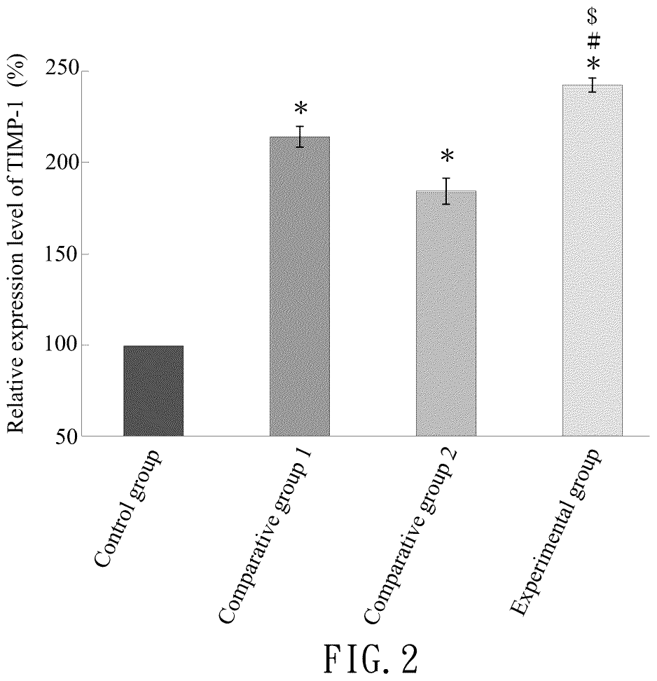

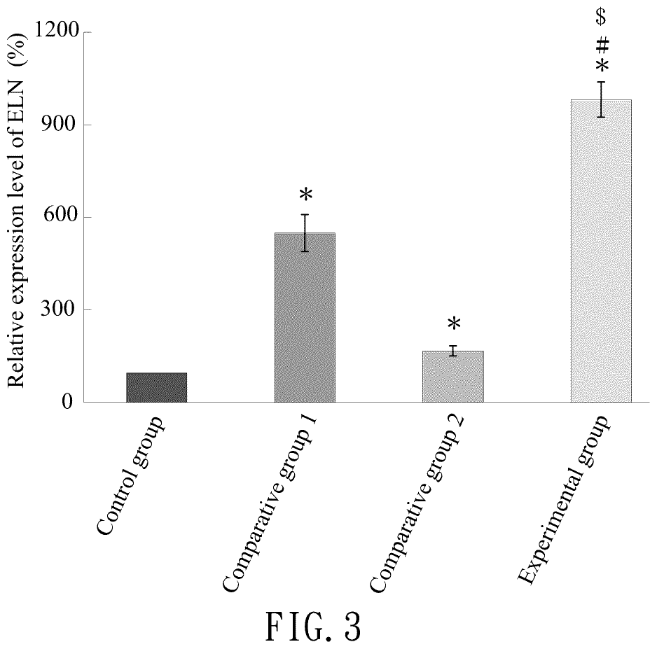

FIGS. 1 to 3 respectively show the relative expression levels of the COL3A1, TIMP-1, and ELN genes determined in each group. As shown in FIGS. 1 to 3, in comparison with the control group, the relative expression levels of the COL3A1, TIMP-1, and ELN genes determined in the comparative groups 1 and 2 showed varying degrees of increase, whereas those determined in the experimental group showed significant increase. To be specific, the relative expression levels of the COL3A1, TIMP-1, and ELN genes determined in the experimental group were significantly higher than those determined in the comparative group 1, and the relative expression levels of the TIMP-1 and ELN genes determined in the experimental group were significantly higher than those determined in the comparative group 2. These results indicate that a composition containing the ethanol-extracted product of Aronia melanocarpa fruit and the Bifidobacterium longum CB108 can exhibit a significantly greater effect on promotion of the expression of the ECM-related genes in dermal fibroblasts compared with the individual use of either the ethanol-extracted product of Aronia melanocarpa fruit or the Bifidobacterium longum CB108. Based on these results, the applicant considers that the composition containing the ethanol-extracted product of Aronia melanocarpa fruit and the Bifidobacterium longum CB108 may exert a synergistic effect on enhancement of ECM production by the dermal fibroblasts, thereby improving skin elasticity. Accordingly, the applicant further conducted the following experiment.

### Example 2. Evaluation of the Effect of Composition Containing Ethanol-Extracted Product of Aronia melanocarpa Fruit and Bifidobacterium longum CB108 on Improvement of Skin Elasticity

### Experimental Subjects

After screening with the exclusion criteria shown in Table 4 below, a total of 9 female subjects aged between 24 years old and 48 years old were enrolled in this trial.

TABLE 4

| Item | Exclusion criteria |
|---|---|
| 1 | Subject with skin disease |
| 2 | Pregnant woman |
| 3 | Breastfeeding woman |
| 4 | Subject who had taken drugs that affected skin condition |
| | thereof during this trial |
| 5 | Subject who had undergone an aesthetic treatment during |
| | this trial |
| 6 | Subject who had been exposed to strong sunlight during this |
| | trial |

### Experimental Procedures

First, the Bifidobacterium longum CB108 postbiotics obtained in section 2 of “General Experimental Materials”, the powder of the ethanol-extracted product of Aronia melanocarpa fruit obtained in section 3 of “General Experimental Materials”, and maltodextrin were mixed according to the amounts as shown in Table 5 below, so as to be formulated into a capsule 1. In addition, the powder of the ethanol-extracted product of Aronia melanocarpa fruit and the maltodextrin were respectively formulated into capsules 2 and 3 as shown in Table 5 below.

TABLE 5

| | Ingredient (mg) | | |
|---|---|---|---|
| | | Powder of ethanol- | |
| | | extracted product | Bifidobacterium |
| | | of Aronia | longum CB108 |
| Capsule | Maltodextrin | melanocarpa fruit | postbiotics |
| Capsule 1 | 150 | 250 | 100 |
| Capsule 2 | — | 500 | — |
| Capsule 3 | 500 | — | — |

Next, all of the female subjects were randomly divided into 3 groups, including an experimental group (n=3), a comparative group (n=3), and a control group (n=3). The female subjects in each group were administered a respective one of the capsules 1 to 3 as shown in Table 6 below, one capsule per day, over a period of 14 days.

TABLE 6

| Group | Capsule |
|---|---|
| Experimental group | Capsule 1 |
| Comparative group | Capsule 2 |
| Control group | Capsule 3 |

Prior to the start of administration (i.e., the 0th day) and on the 14th day after the start of administration, the whole faces of the female subjects in each group were subjected to determination of skin elasticity indices using a Focuskin full face skin tester (purchased from Precision Safety Efficacy Testing Biomed Corporation). A smaller skin elasticity index indicates that a subject has better skin elasticity.

The change rate of skin elasticity of the female subjects in each group was calculated by substituting the skin elasticity indices that determined prior to the start of administration and that determined on the 14th day after the start of administration into the following Equation (1):

$$A = ( B - C ) / B \qquad ( 1 )$$

$$where A = change rate of skin elasticity ( \% )$$

$$B = skin elasticity index determined in each group prior to the start of administration$$

$$C = skin elasticity index determined in each group on the {14}^{th} day after the start of administration$$

### Results

TABLE 7

| Group | Change rate of skin elasticity (%) |
|---|---|
| Control group | −4.5 |
| Comparative group | 6.1 |
| Experimental group | 9.1 |

Table 7 shows the change rate of skin elasticity determined in each group. As shown in Table 7, after the 14-day trial, the change rate of skin elasticity determined in the control group showed a decrease; the change rate of skin elasticity determined in the comparative group showed a slight increase; and the change rate of skin elasticity determined in the experimental group showed a significant increase with the degree of increase reaching approximately 1.5 fold greater than that determined in the comparative group. These results demonstrate that the composition containing the ethanol-extracted product of Aronia melanocarpa fruit and the Bifidobacterium longum CB108 is capable of significantly enhancing the effect on improvement of skin elasticity; by virtue of adding the Bifidobacterium longum CB108, the effect on improvement of skin elasticity is not only superior to that achieved by using the ethanol-extracted product of Aronia melanocarpa fruit alone, but also allows that the required amount of the ethanol-extracted product of Aronia melanocarpa fruit can be substantially reduced.

Summarizing the above test results, it is clear that the composition containing the ethanol-extracted product of Aronia melanocarpa fruit and the Bifidobacterium longum CB108 is capable of exerting the synergistic effect on enhancement of ECM production by the dermal fibroblasts, and hence can effectively improve skin elasticity.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for improving skin elasticity, comprising administering to a subject in need thereof a composition including an ethanol-extracted product of Aronia melanocarpa fruit and Bifidobacterium longum CB108 which is deposited at the Leibniz Institute DSMZ-German Collection of Microorganisms and Cell Cultures GmbH under an accession number DSM 33895 in accordance with the Budapest Treaty.

2. The method as claimed in claim 1, wherein a weight ratio of the ethanol-extracted product of Aronia melanocarpa fruit to the Bifidobacterium longum CB108 ranges from 1:0.01 to 1:100.

3. The method as claimed in claim 1, wherein the composition is a food product.

4. The method as claimed in claim 1, wherein the composition is a pharmaceutical composition.

5. The method as claimed in claim 4, wherein the pharmaceutical composition is administered by a route selected from the group consisting of oral administration and topical administration.

6. The method as claimed in claim 1, wherein the composition is a cosmeceutical composition.
